(19) United States
(12) Patent Application Publication
(10) Pub. No.: US 2006/0229293 A1
(43) Pub. Date: Oct. 12, 2006

(54) Compositions for the treatment of hepatitis C and methods for using compositions for the treatment of hepatitis C

(75) Inventor: Howard S. Lotsof, Staten Island, NY (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: Addiction Research Institute, Inc.

(21) Appl. No.: 11/398,693

(22) Filed: Apr. 6, 2006

Related U.S. Application Data
(60) Provisional application No. 60/668,574, filed on Apr. 6, 2005.
(60) Provisional application No. 60/720,467, filed on Sep. 27, 2005.

(51) Int. Cl. A61K 31/7024; A61K 31/55

(52) U.S. Cl. 514/214.03; 514/23

(57) ABSTRACT

The present invention pertains to a composition comprising Ibogaine, an indole alkaloid, its active salts and its principal metabolite noribogaine, a demethylated form of ibogaine, for the treatment of hepatitis C and hepatitis C related complications, administered in single or multiple dose regimens effective to reduce somatic complaints, liver enzyme values and viral load caused by chronic hepatitis C in patients, and methods of using the same.

### CROSS REFERENCE TO RELATED APPLICATIONS

This application claims priority under 35 U.S.C. § 119(e) to U.S. Patent Application Ser. No. 60/668,574 filed Apr. 6, 2005 and to U.S. Patent Application Ser. No. 60/720,467 filed Sep. 27, 2005, both of which are incorporated herein in their entirety.

### STATEMENT REGARDING SPONSORED RESEARCH OR DEVELOPMENT

“Not Applicable.”

### REFERENCE TO SEQUENCE LISTING

“Not Applicable.”

### BACKGROUND OF THE INVENTION

1. Field of the Invention

The present invention relates to compositions for the treatment of Hepatitis. More specifically, the present invention is directed to a composition including ibogaine and/or noribogaine, and methods of using the same. Still more specifically, the present invention is directed to a composition and the use of a composition comprising one or more of ibogaine, its active salts and principal metabolite noribogaine to treat somatic complaints, elevated liver enzymes and viral load in patients with susceptible hepatitis C.

2. Description of Related Art

Hepatitis C is a member of the group of viruses known as Flaviviridae. The virus was isolated from a blood-borne non-A, non-B viral hepatitis genome and is identified as nonA, nonB. The virus duplicates by RNA replication and is prone to mutation. Approximately 1.8 percent of the population test positive for viral hepatitis C(HCV) antibodies. Chronic HCV affects three to four million individuals in the United States and occurs in greater than 80 percent of the individuals infected with acute HCV. A minority of patients, approximately 15 percent, may clear the virus naturally. The infection, however, is lifelong in the majority of infected individuals and may be life threatening to them.

The virus is principally transferred through blood, though other vectors cannot be ruled out. Subsets of the population including intravenous drug users and intravenous drug users in recovery may have chronic HCV infection rates of 70 to 90 percent. Chronic HCV may progress rapidly or slowly and there is significant diversity of progression. Symptoms for chronic HCV tend to be nonspecific including fatigue, high ALT and AST levels, muscle and joint pain and right-upper-quadrant discomfort or tenderness of the liver. HCV infection is a major risk factor for cirrhosis and liver cancer. An estimated 8,000 to 10,000 fatalities a year are caused by hepatitis C in the United States.

The principal conventional therapy for the treatment of chronic HCV is a therapy of interferon or pegylated interferon in combination with riboviron (as set forth in U.S. Pat. Nos. 6,172,046 and 6,824,768). This therapy leaves much to be desired. Dose regimens, depending on genotype, are 24 or 48 weeks and may be required to be extended or repeated if not efficacious in obtaining a sustained virologic response (SVR) that is the desired outcome for this combination therapy. This therapy, depending on the form and dose of interferon, the genotype of HCV and other factors, demonstrates efficacy of 2 percent to 75 percent. Relapse, depending on study, dose, genotype and other factors, may be as high as 48 percent in subjects who demonstrated a SVR at the completion of combination therapy. Adverse events and side effects, including possible fatal adverse events due to interferon riboviron combination therapy are significant.

Side effects and medication warnings consist of neuropsychiatric events that may include suicide, depression, return to drug abuse, psychosis, hallucinations, bipolar disorders and mania. Other side effects may be bone marrow toxicity including cytopenias, thyroid disorders, hyperglycemia, diabetes; cardiovascular disorders including hypotension, arrhythmia, tachycardia, cardiomyopathy, angina pectoris and myocardial infarction. Additionally, the following signs and/or conditions may be caused or aggravated by interferon riboviron combination therapy: Respiratory failure or collapse including death, fatal and nonfatal ulcerative hemorrhagic/ischemic colitis, abdominal pain, bloody diarrhea, fatal and nonfatal pancreatitis, rheumatoid arthritis, systemic lupus, loss of vision, retinopathy, and retinal hemorrhages. Anaphylaxis may occur and interferon riboviron combination therapy should be considered as a mutagen effecting DNA and as a possible carcinogen.

Interferon riboviron combination therapy is also dangerous to pregnant women directly and indirectly when used as a therapy in the significant other of a pregnant woman or a woman who may become pregnant. This therapy is anticipated to be an abortifacient.

Ibogaine, ibogamine and tabernanthine are among at least 12 alkaloids found in the Tabernanthe iboga plant of Gabon, West Africa. The Gabonese, as well as Africans in other countries on that continent, have used the iboga alkaloids in the Bwiti religion and Mbiri medical societies principally during the last century or two by European accounts.

Isolation and identification of ibogaine was accomplished by Dybowski and Landrin (Compt. rend. ac. sc. 133:748, 1901).

Dr. Robert Goutarel considered by two generations of French chemists to be the “father of ibogaine research” in collaboration with Maurice-Marie Janot filed U.S. Pat. No. 2,813,873 (Nov. 19, 1957) entitled Derivatives of the ibogaine alkaloids. A review of the field followed. Goutarel R, Gollnhofer O & Sillans R, Pharmacodynamics And Therapeutic Applications of Iboga and Ibogaine. (Psychedelic Monographs & Essays, 6:71-111, 1993).

The structure of ibogaine was investigated by Dickel et al (J.A.C.S. 80:123, 1958). The first total synthesis was cited by Buchi et al. (J.A.C.S. 88, 3099, 1966).

Jurg Schneider and Marie McArthur published Potentiation Action Of Ibogaine On Morphine Analgesia (Experiential 12:323-24, 1956) demonstrating a direct effect of ibogaine on opioids. Cerebral Pharmacokinetics Of Tremor-Producing Harmala And Iboga Alkaloids (Zetler et al., Pharmacology 7(4):237-248, 1972) identified noribogaine as well as, reporting on its tremorgenic effects.

Ibogaine's interaction with Substance P and Substance P's effects on the perception of pain have also been considered.

Ibogaine has also been used as an adjunctive agent in psychotherapy and psychoanalysis, and more recently has been described as an agent that may be able to suppress symptoms of dependence or withdrawal from drugs having dependence liability. Discovery of this property of ibogaine led to the issuance of a number of U.S. patents to Howard S. Lotsof, including patents for ibogaine to treat narcotic dependency (U.S. Pat. No. 4,449,096), cocaine and amphetamine abuse (U.S. Pat. No. 4,587,243), alcohol dependency (U.S. Pat. No. 4,857,523), nicotine dependence (U.S. Pat. No. 5,026,697), poly-drug dependence (U.S. Pat. No. 5,152,994) and U.S. Pat. No. 5,591,738 for the treatment of chemical dependence with combinations of iboga and betacarboline alkaloids. These patents initiated two decades of intense research.

Following the disclosures in HS Lotsof's U.S. patents cited earlier, the first publication of clinical reports of the efficacy of ibogaine in treating chemical dependence was by B. Sisko of the International Coalition for Addict Self-Help. Sisko B. (Interrupting Drug Dependency With Ibogaine: A Summary of Four Case Histories. MAPS Bull. (4)2:15-23, 1993). The work of Sheppard followed in which the author published case reports and research of The International Coalition for Addict Self-Help (ICASH), Dutch Addict Self-Help (DASH) and the Amsterdam Squatters movement. Sheppard SG. (A preliminary investigation of ibogaine: case reports and recommendations for further study. J Subst Abuse Treat. (4):379-85, 1994).

In none of the studies reviewed is the effect of iboga alkaloids including ibogaine, its nontoxic salts and/or its principal metabolite noribogaine considered in the treatment of hepatitis C and hepatitis C-related complications.

Based on a review of interferon, riboviron combination therapy it is apparent there is still a need for a less harmful and less toxic therapeutic composition that is useful in the treatment of hepatitis C and hepatitis C-related complications.

### SUMMARY OF THE INVENTION

In accordance with the present invention it has been surprisingly discovered that iboga alkaloids are effective in treating hepatitis C symptoms, including liver swelling, increased ALT, AST and GGT levels and to reduce HCV RNA viral counts.

The present invention thus provides methods of treating somatic complaints, reducing liver enzyme values and reducing viral load of susceptible hepatitis C in animals by administering to a subject a therapeutically effective dose of iboga alkaloids comprising one or more of ibogaine, ibogamine, tabernanthine, their nontoxic salts and/or the converted principal metabolite noribogaine in a dose and time sufficient to accomplish those effects.

The present invention further provides for the administration of effective doses of the prodrug ibogaine, converted to noribogaine and producing plasma levels of ibogaine and/or noribogaine sufficient to reduce somatic complaints, liver enzyme levels and viral RNA in patients.

The present invention also provides effective doses and dose regimens of the prodrug ibogaine, its salts and therapeutic metabolites.

The present invention further provides for the administration of effective doses and dose regimens to be provided in single or multiple doses on a single day or over a period of days in therapeutically effective doses between 0.1 mg/kg and 25 mg/kg of the prodrug ibogaine, converted to noribogaine and producing plasma levels of ibogaine and/or noribogaine sufficient to reduce somatic complaints, liver enzyme levels and viral RNA in patients having chronic hepatitis C.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out herein.

### DETAILED DESCRIPTION OF THE INVENTION

The present invention discloses compositions and methods of treating hepatitis C and hepatitis C-related complications. In general, the compositions of this invention comprise iboga alkaloids comprising one or more of ibogaine, ibogamine, tabernanthine, their nontoxic salts and/or the converted principal metabolite noribogaine in a therapeutic formulation.

While not wishing to be bound by any theory, it is believed that the agents in the compositions are less harmful and less toxic than present anti-hepatitis C therapies.

Methods of using the composition of the present invention and administering to the host a therapeutically effective amount of a composition of this invention are further disclosed. The present invention further provides a method of treating somatic complaints, reducing liver enzyme values and reducing viral load of susceptible hepatitis C, comprising administering to a host a therapeutically effective amount of a composition of this invention.

Another aspect of the present invention is the shortness of the treatment period, as compared to existing therapies, in that the present treatment period may be a single day or a period of approximately two weeks.

The basis of the present invention is the finding that certain plants contain iboga alkaloids that assist in the treatment of hepatitis C and hepatitis C-related complications. This offers the advantage of allowing therapeutically effective doses of natural agents to be used as compared to the dose of synthetic substances that would be required in order to achieve the same or similar therapeutic effect. Until this invention, it was not known that natural agents extracted from the Tabernanthe iboga plant of Gabon, West Africa, could be combined into one single formulation that would possess the ability to treat hepatitis C and hepatitis C-related complications.

Unless defined otherwise, all technical and scientific terms used herein have the same meaning as commonly understood by one of ordinary skill in the art to which this invention pertains. Although any methods and materials similar or equivalent to those described herein can be used in the practice of the present invention, the preferred methods and materials are described.

The effective amount or effective dose is an amount of the composition to be administered to the host that treats hepatitis C and hepatitis C-related complications. Suitable doses of a composition can be determined readily by various methods known to one skilled in the art, including generating an empirical dose-response curve, and other methods used in the pharmaceutical sciences.

The agents used in the compositions of this invention may be provided in the form of pure substances, or as root bark of the natural plant containing the natural agents in concentrations between about 1 to 6 percent of which approximately fifty percent is ibogaine, or as concentrated plant extracts containing the natural agents in concentrations between about 5 to 40 percent of which one half is ibogaine. Doses of the root bark or total alkaloid extract would be extrapolated to correspond to doses of purified ibogaine in keeping with the dose recommendations and regimens for purified ibogaine and associated alkaloids as described in the present invention. The amount of agent contained in a composition of this invention will depend in part on the desired results of the treatment, the stage of hepatitis C, its associated complications, and/or the health of the patient.

Another embodiment of the present invention includes improved methods for using the agents. It was discovered that various methods of administering the present invention to a host achieve therapeutically effective results, thus allowing therapeutically effective doses of agents to be administered to patients that suffer from various medical conditions, for example, conditions that make oral administration and digestion difficult. Until this invention, it was not known that the agents of the present invention could be administered by methods that would treat hepatitis C and hepatitis C-related complications in the host.

The present invention thus provides methods of treating hepatitis C and hepatitis C-related complications comprising administering a composition of this invention to a host in need of therapy. The doses, routes of administration, and carriers and/or adjuvants used may vary based on the view of known procedures for treatment of hepatitis C and hepatitis C-related complications or the delivery of any manner of drug product known to those familiar with the art.

One feature of the method of using the invention is that the composition can be administered in the form of a tablet, capsule or other pharmacologically appropriate carrier, in a parenteral solution, in a suppository, in a rectal solution, in the form of a tea, or in the form without plant material in a tablet, capsule, transdermal technology or other pharmacological carrier, in a parenteral solution, or in a suppository which contains at least one of the agents comprising iboga alkaloids.

The compositions of this invention may also be administered as a solution and other oral or parenteral administration can be used. For example, a compound with poor solubility in acidic media may show poor or erratic bioavailability when absorbed orally. Further, intravenous administration requires that a drug be administered in a soluble form. Compounds that are intended for oral administration but are susceptible to rapid degradation at low pH (i.e. gastric acids) will likely require protection from low pH environments like the stomach. Protection can often be afforded by administering the drug in a dosage form with an acid-resistant coating. Thus, while it is possible to administer the compositions of this invention alone, the compositions may also be administered as part of a formulation. For oral administration, the compositions of this invention can be used in the form of tablets, capsules, granules, powders, lozenges, syrups, elixirs, solutions, suspensions, and the like, in accordance with standard pharmaceutical practice. A dried extract can be compounded into tablets, capsules, or other solid-dosage form. A solubilized liquid formulation can be combined with syrup or other agent to formulate suspensions, solutions, elixirs, or tinctures to improve the taste, potency, or shelf life.

For parenteral administration, which includes intramuscular, intraperitoneal, subcutaneous and intravenous use, sterile solutions of the agents are usually prepared, and the pH of the solutions are suitably adjusted and buffered.

Carriers useful in formulating the preparations are commonly used pharmaceutically acceptable non-toxic carriers such as gelatin, lactose sodium citrate, salts of phosphoric acid, starch, magnesium stearate, sodium lauryl sulfate, talc, polyethylene glycol, etc. The carrier may be used with other additives such as diluents, binders, buffer agents, preservatives, sweetening agents, flavoring agents, glazes, disintegrators, coating agents emulsifying agents, suspending agents, etc.

The dosage regimen may be regulated according to the potency of the individual agents utilized in the compositions of this invention, the mode of administration, and the needs of the host depending on factors such as the degree and severity of the disease state and age and general condition of the host being treated. Dosing ranges from 0.1 to 25 milligrams of the composition of the present invention per kilogram of body weight, once or multiple times daily, for one day to four weeks or longer depending upon the severity and length of hepatitis C infection and the response of the patient.

The present invention also provides methods of treating hepatitis C and related complications in the significant other or sexual partner of a pregnant female without having an observable toxic effect on the fetus.

A further embodiment of the present invention provides for the treatment of hepatitis C symptoms, including liver swelling, increased liver enzyme levels, including γ-glutamyl transferase (GGT), Aspartate aminotransferase (AST), Alanine aminotransferase (ALT) and Alkaline Phosphatase levels, and for the reduction in HCV RNA viral counts. Elevated levels of GGT, ALT, AST and Alkaline Phosphatase indicate injury or trauma to the liver. Conversely, reduced levels of GGT, AST, ALT and Alkaline Phosphatase indicate reduced trauma or injury of the liver.

Another embodiment of the present invention is that it can be effective in a chemically dependent population—a population which has greater tendency to be susceptible to hepatitis C infection.

Another embodiment of the present invention is that it can concurrently treat signs and symptoms of hepatitis C infection and chemical dependence disorders.

### SPECIFIC EXAMPLES

The following embodiments are for illustrative purposes only and are not intended nor should they be interpreted to limit the scope of the application.

### Example 1

A thirty-three year old male diagnosed as HCV positive and using ½ gram of heroin per day was administered 25 mg/kg of ibogaine HCl. Following the administration of ibogaine, heroin use ceased along with swelling of the liver and pain in the area of the liver.

### Example 2

A twenty-six year old male testing positive for HCV and dependent on heroin and methadone self-administered 14 mg/kg of ibogaine HCl. AST was reduced from pretreatment level of 201 to post treatment level of 25. ALT was reduced from pretreatment level of 410 to post treatment level of 50. GGT level was reduced from 155 to 33.

### Example 3

A sixty year old male testing positive for HCV RNA genotype 1, administered the following dose regimen of ibogaine HCl. Subject weighed 79 kg. Doses administered were as total doses and not mg/kg. Day 1: 10 mg ibogaine HCl. Day 2: 20 mg ibogaine HCl. Day 3: 20 mg ibogaine HCl. Day 4: 30 mg ibogaine HCl. Day 5: 50 mg ibogaine HCl. Day 6: 75 mg ibogaine HCl. Day 8: 100 mg ibogaine HCl. Day 10: 150 mg ibogaine HCl. Day 14: 300 mg ibogaine HCl. HCV RNA IU/mL was reduced from 780,000 to 644,000. Pretreatment Alkaline Phosphatase was 99, AST was 103 and ALT 195. Post treatment Alkaline Phosphatase 88, AST 89 and ALT 127. An additional 250 mg of ibogaine HCl further reduced HCV RNA IU/mL to 384,000. A final dose within this regimen of 250 mg ibogaine was administered reducing the HCV RNA IU/mL to 154,000.

### Example 4

A forty-two year old female weighing 160 lbs tested positive HCV RNA genotype 3. RNA IU/mL was 12,600,000. Subject was administered a total of 27 mg/kg of ibogaine HCl over a period of 48 hours in the following regimen: 2 mg/kg, 2 mg/kg, 2 mg/kg, 2 mg/kg, 2 mg/kg, 2 mg/kg, 12 mg/kg, and 3 mg/kg. HCV RNA IU/mL was reduced to 50,100. Prior to ibogaine therapy patient's urine was dark and stool light. Post treatment color of urine and stool were normal.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

I claim:

1. A composition comprising one or more of ibogaine, ibogamine, tabernanthine, their nontoxic salts and/or the converted principal metabolite noribogaine in a therapeutically effective concentration for the treatment of hepatitis C or hepatitis C-related complications.

2. The composition of claim 1, further comprising a pharmaceutically acceptable carrier, excipient or dilutant.

3. The composition of claim 1, wherein said nontoxic salt is selected from one or more of hydrochloride, sulfate, phosphate, tannate, acetate and tartrate.

4. The composition of claim 1, in the form of a capsule, tablet, liquid or powder.

5. The composition of claim 1, wherein said ibogaine, ibogamine, tabernanthine, their nontoxic salts and/or the converted principal metabolite noribogaine is in the form of the botanical plant in the form of root bark or concentrated plant extracts between 1 to 40% by weight.

6. The composition of claim 1, wherein said ibogaine, ibogamine, tabernanthine, their nontoxic salts and/or the converted principal metabolite noribogaine are administered in a dose from 0.1 to 25 milligrams per kilogram of body weight.

7. A method for treating hepatitis C or hepatitis C-related complications which comprises administering a pharmaceutically effective amount of a composition comprising ibogaine, ibogamine, tabernanthine, their nontoxic salts and/or the converted principal metabolite noribogaine to a human or mammal.

8. The method of claim 7, wherein said ibogaine, ibogamine, tabernanthine, their nontoxic salts and/or the converted principal metabolite noribogaine are administered in a dose from 0.1 to 25 milligrams per kilogram of body weight.

9. The method of claim 7, wherein said composition is administered in a tablet, capsule, pharmacological carrier, parenteral solution, transdermal technology, suppository, or liquid.

10. The method of claim 7, wherein said composition is admixed with binders, fillers or other inert ingredients.

11. The method of claim 7, wherein said composition is administered in a single dose from 0.1 to 25 mg/kg of body weight.

12. The method of claim 7, wherein said composition is administered in a plurality of doses, each dose from 0.1 to 25 mg/kg of body weight.

13. The method of claim 12, wherein said composition is administered in said plurality of doses over the course of one day to four weeks or longer.

14. A method for treating, reducing elevated liver enzyme levels, which comprises administering a pharmaceutically effective amount of a composition comprising ibogaine, ibogamine, tabernanthine, their nontoxic salts and/or the converted principal metabolite noribogaine to a human or mammal.

15. The method of claim 14, wherein said ibogaine, ibogamine, tabernanthine, their nontoxic salts and/or the converted principal metabolite noribogaine are administered in one or more doses from 0.1 to 25 milligrams per kilogram of body weight.

16. A method for treating, reducing swelling of the liver, which comprises administering a pharmaceutically effective amount of a composition comprising ibogaine, ibogamine, tabernanthine, their nontoxic salts and/or their converted principal metabolite noribogaine to a human or mammal.

17. The method of claim 16, wherein said ibogaine, ibogamine, tabernanthine, their nontoxic salts and/or the converted principal metabolite noribogaine are administered in one or more doses from 0.1 to 25 milligrams per kilogram of body weight.

18. A method for treating, reducing the perception of pain in the liver, which comprises administering a pharmaceutically effective amount of a composition comprising ibogaine, ibogamine, tabernanthine, their nontoxic salts and/or the converted principal metabolite noribogaine to a human or mammal.

19. The method of claim 18, wherein said ibogaine, ibogamine, tabernanthine, their nontoxic salts and/or the converted principal metabolite noribogaine are administered in one or more doses from 0.1 to 25 milligrams per kilogram of body weight.

20. A method for treating, reducing hepatitis C RNA levels, which comprises administering a pharmaceutically effective amount of a composition comprising ibogaine, ibogamine, tabernanthine, their nontoxic salts and/or the converted principal metabolite noribogaine to a human or mammal.

21. The method of claim 20, wherein said ibogaine, ibogamine, tabernanthine, their nontoxic salts and/or the converted principal metabolite noribogaine are administered in one or more doses from 0.1 to 25 milligrams per kilogram of body weight.

22. A method for treating, reducing or preventing elevated levels of the liver enzymes comprising one or more of γ-glutamyl transferase (GGT), Aspartate aminotransferase (AST), Alanine aminotransferase (ALT) and Alkaline Phosphatase, which comprises administering a pharmaceutically effective amount of a composition comprising ibogaine, ibogamine, tabernanthine, their nontoxic salts and/or the converted principal metabolite noribogaine to a human or mammal.

23. The method of claim 22, wherein said ibogaine, ibogamine, tabernanthine, their nontoxic salts and/or the converted principal metabolite noribogaine are administered in one or more doses from 0.1 to 25 milligrams per kilogram of body weight.
